(19) United States
(12) Patent Application Publication
(10) Pub. No.: US 2005/0229281 A1
(43) Pub. Date: Oct. 20, 2005

(54) Hygienic eyes cover

(76) Inventor: Jerome Glasser, Maplewood, NJ (US)

Correspondence Address: JEROME GLASSER, 16 Sunset Terrace, Maplewood, NJ, 07040, US

(21) Appl. No.: 10/827,060

(22) Filed: Apr. 19, 2004

(51) Int. Cl. A61F 9/00

(52) U.S. Cl. 2/15

(57) ABSTRACT

Inexpensively manufactured device permits a eye mask, eyes cover, face shield or mask, sleep shade or sleep mask, light shade, blindfold, eye patch for a single eye and/or other such facial cover, to hold a single or a grouping of hygienic plastic, paper or other hygienic material in a removeable fashion so that easy cleaning can be effected after use and/or the hygienic article can be discarded so that the post-use impure material does not contact again the same or a different user's face. The device of this invention may also be impregnated with a scent and/or a cream, gel or other element to impart a substance or treatment onto eyes and/or face over a prolonged period.

### FIELD OF THE INVENTION

The present invention relates to improvements in the field of eyes covers, light shields, blindfolds, eye patches, face masks, sleep masks and/or sleep or eye shades.

### BACKGROUND OF THE INVENTION

Prior art discloses some eye covers, many of which disclose an outright attempt to prevent the eye cover from coming in contact with a user's eyes. None addresses the problem of the unsanitary nature of the repeated use of the same eyes cover by one person without properly cleaning the cover often. The face, eyes particularly, needs to be kept clean, and thus, the nature of covers placed on the face and eyes is such that they become infested with germs during first use. Therefore, there is a significant need for ensuring that a face mask is not used a second time before cleaning. This is considered a burdensome chore by most users who typically take for granted the need for cleanliness.

Despite the fact that conventional, traditional eye covers do not stay clean and actually become filled with harmful bacteria quite quickly, the most widely marketed eyes covers are foam-filled and are not designed with the intention that they be readily and easily washed. This contention is supported by the fact that, when cleaning is attempted, the eyes covers do not dry quickly, and, furthermore, are not sold with washing instructions.

Moreover, none of the prior art suggests the problem of the inability, for the sake of good hygiene, for one user to share an eye cover with another user.

Prior art in the eye covering field includes U.S. Pat. No. 6,571,799 (2003) to Daly which discloses protective eye shades which have a hook-and-loop fastening system that is primarily designed for children and relates to methods and devices for attaching a face mask to the head.

U.S. Pat. No. 6,320,094 (2001) to Arnold discloses a disposable eye patch having an adhesive layer suitable for a medical patient and which is separated to be used for one single eye at a time.

U.S. Pat. No. 6,293,900 (2001) to Bove, et al. discloses a face mask incorporating magnetic fields having a perimeter cushion serving to hold the eye mask away from the user's eyes and lashes.

U.S. Pat. No. 5,940,886 (1999) to Smith relates to face mask sleep shades seemingly having open and closed states operational within the mask itself.

U.S. Pat. Nos. 5,673,432 (1997) and 5,435,006 (1995) and 4,872,217 (1989) to Kityama disclose eye masks which do not contact the eyelids of a user.

U.S. Pat. No. 4,411,263 (1983) discloses yet another hook-and-loop fastening system that is primarily designed for children and relates to methods and devices for attaching a face mask to the head.

U.S. Pat. No. 4,162,542 (1979) to Frank discloses a sun goggle having no apparent means of being secured to the uses face.

U.S. Pat. No. 2,643,382 (1953) to McLeod discloses a single eye patch having holes.

U.S. Pat. No. 2,527,027 (1950) to Mull discloses a sun goggle device.

U.S. Pat. No. 2,305,080 (1942) to Hemphill seems to disclose the model for the eyes cover on which the traditional, conventional eyes cover is based and makes no reference to maintaining hygienic properties for sanitary use.

U.S. Pat. No. 589,307 (1897) to Seffer discloses a “hoodwink” which seems to disclose two rigid, opaque elements curved to conform to the general shape of the human eyes and which are slidably attached along a ribbon or string.

While some specialty eyes covers do achieve a certain, moderate success in attempts to solve one, perhaps two or even an ambitious three of the many factors that detract from the overall positive experience of eyes cover use including: the inability to function well as light blockers; the inability to comfortably conform to a user's face; excessive weight that uncomfortably presses down on a user's face; discomfort and inability to stay on a user's face arising from the securing method; nevertheless, none has ever addressed one of the primary deficiencies in eyes covers, that being the unhygienic nature of repeated use of previously invented embodiments.

### SUMMARY AND OBJECTS OF THE INVENTION

The invention of this disclosure allows for the production of an inexpensive, long-lasting, reusable sleep mask or eye cover, and one which can be shared among multiple users and which provides the highest level of hygiene. The reason for this is that the part of the eye cover which primarily comes in contact with the face is disposable. Another benefit of this invention is that it generates new business for manufacturers of sanitary paper and/or plastics.

The invention of this disclosure provides a paper-holding element which is contoured to comfortably fit a person's face and which is designed and manufactured to accept and hold in place at least one, but preferably a grouping of sanitary tissues, each of which is disposable after a one-time use.

Accordingly, a primary object of this invention is to provide device which can be inexpensively produced and which can serve as a cover for the eyes to hinder light from disturbing a person's attempt at rest and/or sleep.

Another object of this invention is to provide a device which will avoid the build-up of germs, dirt and other impurities injurious to one health by having an removable surface that comes in contact with the face, and which may be discarded, if so desired.

Yet another object of this invention is to provide a tissue which can be impregnated with a substance that can contribute to the rejuvenation of facial skin cells and which can be worn, especially during resting or sleeping periods, covering the eyes and facial area, particularly eyelids and eye corners, to prevent and/or retard the development of “crows feet”.

Yet still another object of this invention is a device which can uniformly apply a sunscreen or some other form of face or skin care treatment around the facial area.

Yet another still further object of this invention is a device which can be impregnated with a scent to which a user can be exposed while using the invention of this dislcosure. The scent can be imparted via its impregnation in the main body of the invention, or via a tissue or tissues held in place by the main body and released over time.

Yet still another, further, object of this invention is a device which can be used as an advertising specialty product to promote a product and/or service

### BRIEF DESCRIPTION OF THE DRAWINGS

The invention is herein described, somewhat diagrammatically and by way of example only, with reference to the accompanying drawings, wherein:

FIG. 1 is a frontal view of a preferred embodiment of the interior-facing side of an eye cover having an elastic-type securing band, and suited to receive hygienic paper, either in single sheets or in a “cartridge” format.

FIG. 2 is a frontal view of the cover material not in contact with a user's face when used.

FIG. 3 is a frontal view of a single sheet of hygienic paper or plastic.

FIG. 4 is a grouping of sheets of hygienic paper or plastic suitable to be placed inside the eye cover.

FIG. 5 discloses a grouping of sheets of hygienic paper or plastic collected and held as a single grouping in a “cartridge” format suitable to be easily placed inside the eye cover.

FIG. 6 demonstrates how the cartridge is hygienically wrapped for sanitary purposes.

FIG. 7 discloses a grouping of sheets of hygienic paper or plastic suitable to be placed in the eye covers, and indicates the path which is followed to put the sheets in place.

FIG. 8 is a perspective view disclosing the sheets approximately ⅔^{rdS} of the way fully placed inside the eye covers.

FIG. 9 discloses a frontal view of a resealable sanitary holder for the invention.

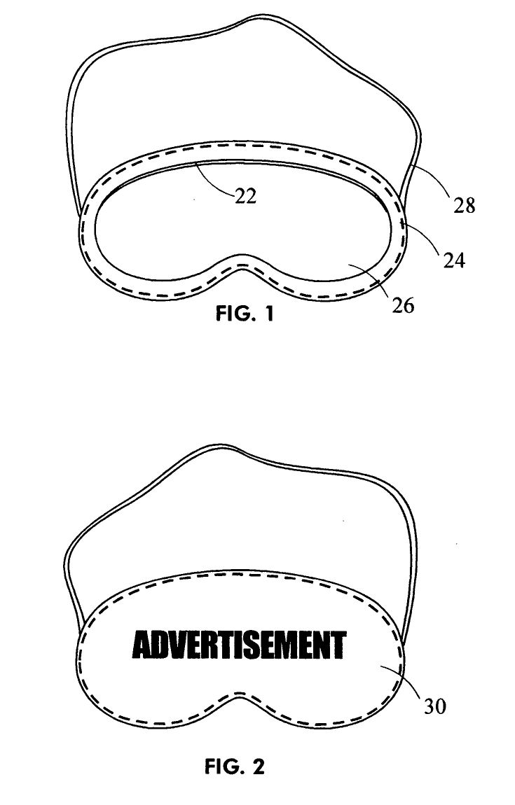

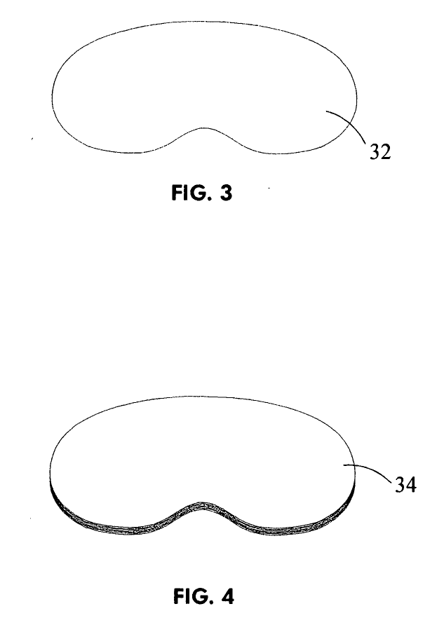

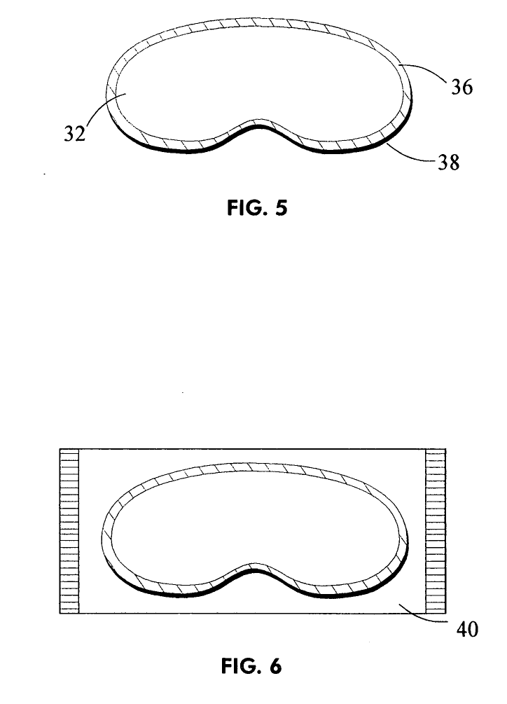

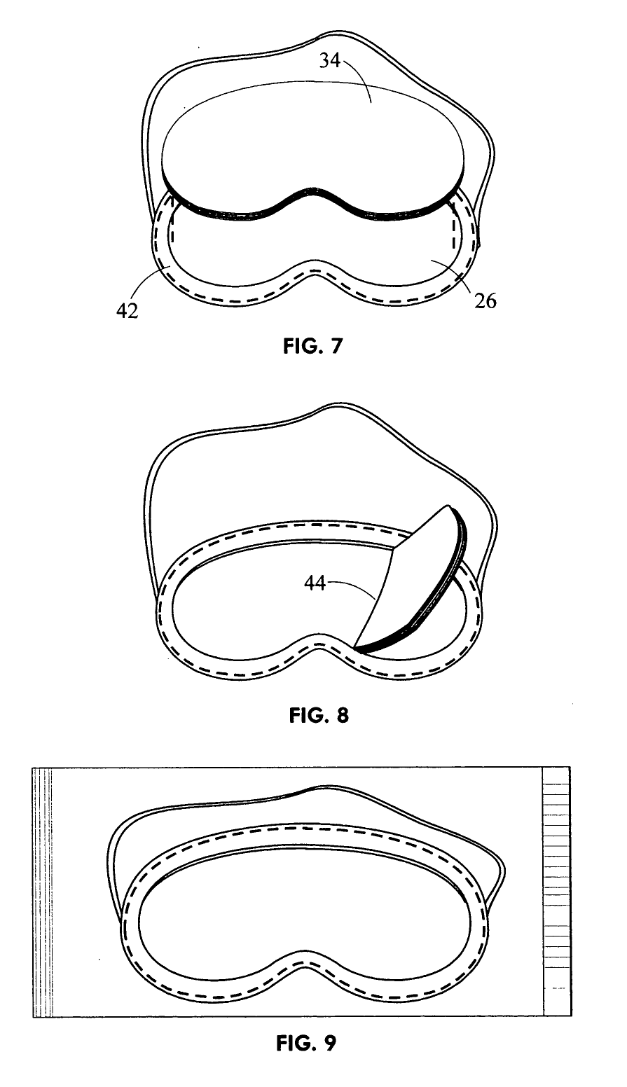

REFERENCE NUMERALS IN DRAWINGS

22 Holding Lip Edge
24 Seam
26 Main Cover Interior
28 Headband
30 Main Cover
32 Hygienic Sheet
34 Hygienic Sheet Grouping
36 Cartridge Top
38 Cartridge Side
40 Hygienic Cartridge Wrapping
42 Main Cover Wrapping

### DESCRIPTION OF THE PREFERRED EMBODIMENT

There is shown a Main Cover 30 having an essential oval shape with an interiorly formed indentation in order that the shape of Main Cover 30 will avoid the uncomfortable abutment of the bridge of the user's nose. Main Cover 30 has Holding Lip Edge 22 formed by taking Main Cover 30 and folding it interiorly upon and over itself and then covering it with Main Cover Wrapping 42. Seam 24 is stitched to maintain the shape of Holding Lip Edge 22, which thereby defines an interior compartment suitable for receiving the introduction of Hygienic Sheet Grouping 34 which may be held together as a grouped unit by a thin, plastic Cartridge Side 38. The outward-facing (i.e. non-face-touching) side of Main Cover 30 may be silk-screen imprinted for advertising promotion.

A plastic Hygienic Cartridge Wrapping 40 wrapping is provided to maintain a high-level of hygiene for the eye cover, as well as the Cartridge.

After Hygienic Sheet Grouping 34 is removed from Hygienic Cartridge Wrapping 40 and is placed inside Holding Lip Edge 22, the invention of this disclosure is ready for use. After use of the invention, Hygienic Sheet 32 is extracted from Sheet Grouping 34 using fingers and then disposed of thereby leaving a clean, sanitary, new Hygienic Sheet 32 in the invention ready for the next, and now hygienic use.

A storage container may be provided for keeping the invention even more sanitary.

The above and other objects, features and advantages of the present invention will become apparent from the following description thereof to be read in connection with the accompanying drawings.

While the above description contains many specificities, these should not be construed as limitations on the scope of the invention, but rather as an exemplification of one preferred embodiment thereof. It is apparent that changes and modifications can be made and equivalents substituted without departing from the invention. Other variations are possible. Accordingly, the scope of the invention should be determined not by the embodiments illustrated, but by the appended claims and their legal equivalents.

1. A device for hygienically covering the eyes:
at least a first element;
at least one second planar element;
at least a means for holding said at least one second planar element in contact with said at least a first element.
